(19) United States
(12) Patent Application Publication
(10) Pub. No.: US 2024/0093263 A1
(43) Pub. Date: Mar. 21, 2024

(54) METHODS OF ATTACHING PROBES TO MICROORGANISMS AND METHODS OF USE THEREOF

(71) Applicant: REGENTS OF THE UNIVERSITY OF MINNESOTA, Minneapolis, MN (US)

(72) Inventor: Abdennour Abbas, St. Paul, MN (US)

(21) Appl. No.: 18/128,048

(22) Filed: Mar. 29, 2023

Related U.S. Application Data
(62) Division of application No. 16/073,092, filed on Jul. 26, 2018, now abandoned.
(60) Provisional application No. 62/287,894, filed on Jan. 27, 2016.

(51) Int. Cl. C12Q 1/06; C12Q 1/04; C12Q 1/10; G01N 33/569

(52) U.S. Cl. CPC C12Q 1/06; C12Q 1/04; C12Q 1/10; G01N 33/569; G01N 33/56916

(57) ABSTRACT

Methods that include combining a probe with a composition including a microorganism, wherein the probe includes a targeting portion that interacts with a phylogenetic surface marker (PSM) on the microorganism to at least partially coat the microorganism with the probe.

### PRIORITY

This application claims priority to U.S. Provisional Application No. 62/287,894 filed on Jan. 27, 2016 entitled METHODS OF COATING MICROORGANISMS WITH NANOPARTICLES AND METHODS OF USE THEREOF AND COMPOSITIONS, the disclosure of which is incorporated herein by reference thereto.

### GOVERNMENT FUNDING

This invention was made with Government support under contract number 1605191 awarded by the National Science Foundation (NSF). The Government has certain rights in this invention.

### BACKGROUND

Many industries, including the food industry, food safety agencies, drug manufacturers, environmental monitoring, and diagnostic laboratories continue to express a need for a rapid, cost-effective microbial detection technology capable of providing reliable results in less than 2 hours.

### SUMMARY

Disclosed herein are methods that include combining a probe with a composition including a microorganism, wherein the probe includes a targeting portion that interacts with a phylogenetic surface marker (PSM) on the microorganism to at least partially coat the microorganism with the probe.

Also disclosed are methods of analyzing a microorganism in a composition, the method including combining a probe with a composition comprising the microorganism, wherein the probe includes a targeting portion and an active portion wherein the targeting portion interacts with a phylogenetic surface marker (PSM) on the microorganism to at least partially coat the microorganism with the probe; and analyzing the at least partially coated microorganism based on one or more properties of the active portion of the probe, wherein the targeting portion and the active portion can but need not be the same material.

Also disclosed herein are compositions that include a microorganism coated with a probe the probe including a targeting portion, wherein the targeting portion interacts with one or phylogenetic surface markers (PSMs) on the surface of the microorganism.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 is a schematic illustration showing a modification of disulfide bridges present in proteins on all microorganisms and the combination thereof with gold containing nanoparticles.

FIG. 2 is a schematic illustration showing a modification of chitin present in all fungi and the combination thereof with gold containing nanoparticles.

FIG. 3 is a schematic illustration showing lipoteichoic acid present on all gram positive bacteria and the combination thereof with hydroxyapatite containing nanoparticles.

FIG. 4 shows scanning electron microscopy (SEM) images at various scales (10 JAM, 1 μm, and 100 nm) and corresponding energy dispersive X-ray spectroscopy (EDX) maps of the nanocoated fungi which shows gold (Au)

FIGS. 5A, 5B, 5C and 5D show a photograph (FIG. 5A) and the nanoparticle aggregation percentage versus the E. coli concentration (cfu/mL) for the samples of E. coli in water (FIG. 5C) and a photograph (FIG. 5B) and the nanoparticle aggregation percentage versus the E. coli concentration (cfu/mL) for the samples of E. coli in milk (FIG. 5D).

FIG. 6A shows an image of a control (with no microorganisms) and FIG. 6B shows an image of a sample containing bacteria.

FIGS. 7A, 7B and 7C show images of AuNP solution mixed with either fungi (Mucor) (FIG. 7A) or bacteria (E. coli) (FIG. 7B) showing the change in color after addition of a reducing agent TCEP; and FIG. 7C shows a surface-enhanced Raman spectra of a mixture of fungi (Mucor) and AuNPs before (upper trace) and after (lower trace with inset) addition of a reducing agent TCEP. The peak at 317 cm⁻¹ is assigned to Au—S bonds.

FIGS. 8A, 8B, 8C, 8D and 8E show images of AuNP solution mixed with fungi (Mucor) after deacetylation with 50% NaOH (FIG. 8A). FIG. 8B shows the images when a large piece of fungi is used (the yellowish substance (Mucor) turns dark by assembling the nanoparticles on its surface after 5 minutes); and the solution becomes transparent after all the nanoparticles are assembled on the fungal surface. FIG. 8C shows an image of an AuNP solution; FIG. 8D shows an image of after the deacetylation of Lactobacillus (bacteria) with AuNP and FIG. 8E shows deacetylated Mucor (fungi) with AuNP.

FIGS. 9A to 9I show scanning electron microscopy (SEM) (FIGS. 9A, 9B, 9E and 9F), transmission electron microscopy (TEM) (FIGS. 9D, 9G, 9H and 9I) and energy-dispersive X-ray spectroscopy mapping (EDX) (FIG. 9C) of E. coli cells coated with gold nanoparticles. FIGS. 9J to 9Q show SEM (FIGS. 9J, 9K and 9N), TEM (FIGS. 9M, 9P and 9Q) and EDX (FIGS. 9L and 9O) of Lactobacillus coated with gold nanoparticles. FIGS. 9R to 9Y show SEM (FIGS. 9R, 9S, 9V and 9W), TEM (FIGS. 9U, 9X and 9Y) and EDX (FIG. 9T) of spores from Mucor coated with gold nanoparticles.

FIGS. 10A to 10D are SEM images of bacteria E. coli showing the binding of gold nanoparticles to bacterial pili.

FIGS. 11A to 11F show SEM (FIGS. 11A, 11B and 11C) and TEM (FIGS. 11D, 11E and 11F) images of nanocoatings peeled off of the cells.

FIGS. 12A to 12E shows the concept of homogenous microbial screening by cell nanocoating using either (1) fluorescence emission and quenching by AuNPs or (2) light absorption by plasmonic coupling of localized surface plasmon resonance on AuNPs (FIG. 12A); the effect of the reducing agents TCEP and BME on the aggregation of gold nanoparticles at different pH conditions. The concentrations that do not cause nanoparticle aggregation are used for microbial screening assays (FIG. 12B); Image of AuNP solution with different concentrations of bacteria E. coli. The image was taken 2 min after mixing the nanoparticles with reduced bacteria (FIG. 12D); UV-visible spectra of the different samples showed in Figure “c”. Single nanoparticle solutions exhibit a maximum absorption peak at around 525 nm. During cell nanocoating, a second peak appears and grows in intensity at around 600 nm. This peak is caused by plasmonic coupling of assembled gold nanoparticles, and reflects the deposition of the nanoparticles on the microbial surface. The correlation between the microbial concentration and the absorption intensity is provided on the right. A linear range can be identified between 10³ and 10⁷ cfu.mL−1 (r²=0.96) (FIG. 12D); Detection of fungi Mucor (top graphic) and bacteria E. coli (bottom graphic) using fluorescence quenching of Rhodamine 6G. The linear ranges are from 250 to 10⁴ cfu.mL−1 for Mucor (r²=0.98), and 0 to 10⁵ cfu.mL−1 for E. coli (r²=0.99) (FIG. 12E); and FIG. 12F shows the effect of the reduction of the concentration of the gold nanoparticles on the visual reading of the microbial load.

FIG. 13 schematically illustrates an example of the general concept of cell coating with probes.

FIG. 14 schematically illustrates the combination of cell coating with probes and separation utilizing magnetic beads.

FIG. 15 schematically illustrates microbial identification using cell coating with probes with a signal amplification system.

The figures are not necessarily to scale. Like numbers used in the figures refer to like components. However, it will be understood that the use of a number to refer to a component in a given figure is not intended to limit the component in another figure labeled with the same number.

### DETAILED DESCRIPTION

According to a recent report published in December 2015 by the world health organization, foodborne diseases worldwide cause 420,000 deaths (6000 in the US) and over 600 million (almost 1/10) people to fall ill every year, with an overall economic impact as high as $77 billion per year in the US alone. Instantaneous microbial detection would significantly reduce these numbers, but also reduce food loss by allowing rapid detection of spoilage agents and early intervention to delay deterioration. According to a report published in February 2014 by the USDA, food loss totaled 31% of the available food supply in United States, which represents a food security problem. The estimated total value of this loss was $161.6 billion in 2010. Instantaneous microbial detection along the food supply chain would substantially help implement preventive actions and reduce food poisoning, waste and costly recalls. The impact extends to early detection of plant and tree diseases for precision agriculture and forestry.

In addition to the benefit to public health, food testing represents a rapidly growing market. The testing market is projected to reach USD 16.1 Billion by 2020.

The food industry especially is interested in rapid and cost-effective microbial detection technology capable of providing reliable results in less than 2 hours. The 2-hour time frame can be important as food producers, for example, seek to control the quality and safety of their products on-site before shipment. Once the product is released, any undetected contamination or spoilage would lead to significant costs due to recalls, food waste, food poisoning or negative impact on the product perception and consumer trust.

Although many commercially available detection technologies are labeled for “rapid microbial detection”, they often require 12 to 48 hours of enrichment before detection. However, because of the time frame, the food industry prefers to rely on much cheaper cell culture and microscopic identification.

In addition to conventional cell culture and count, the evaluation of the microbial content in food samples can be performed by the detection of the presence of adenosine triphosphate (ATP) using bioluminescence (Bottari, B., M. Santarelli, and E. Neviani, Determination of microbial load for different beverages and, oodsmff by assessment of intracellular ATP. Trends in Food Science & Technology, 2015. 44(1): p. 36-48). A number of commercial ATP-bioluminescent kits and equipment are commercially available. However, their use is limited due to the need of cell enrichment to reach a detectable signal.

Spoilage of food by fungi is not only a quality concern, but also a food safety issue. A large number of mold species are highly toxigenic due to the production of mycotoxins in foods. Despite their importance in food spoilage and public health, the detection of fungi has not seen significant technological development as compared to the detection of bacteria. The main method currently used by the food industry for yeasts and molds detection is cell plating and incubation for 5 to 7 days followed by colony count. Some commercial products such as 3M PETRIFILM® claim to cut incubation times from five days to 48 to 72 hours. Other commercial products are based on flow cytometry (CHEMUNEX® from bioMerieux (Etoile, France)), detection of CO2 produced by the fungi (eg. BioLumix (Neogen Corporation, Lansing, MI)), or use DNA amplification for a specific fungi sequence (eg. BAX® system from Dupont (Wilmington, DE)). All those instruments require processing time from 24 to 72 hours. The majority of techniques developed for the detection of gram-positive bacteria are developed for clinical application and thus they are not suitable for use on food samples (Aitken, S. L., et al., Real-world performance of a microarray-based rapid diagnostic for Gram-positive bloodstream infections and potential utility for antimicrobial stewardship. Diagnostic Microbiology and Infectious Disease, 2015. 81(1): p. 4-8; and Siu, G. K. H., et al., Performance Evaluation of the Verigene Gram-Positive and Gram-Negative Blood Culture Test for Direct Identification of Bacteria and Their Resistance Determinants from Positive Blood Cultures in Hong Kong. PLoS ONE, 2015. 10(10): p. e0139728). A number of other techniques have been reported in scientific literature including the use of impedimetric biosensors (Etayash, H., et al., Impedimetric Detection of Pathogenic Gram-Positive Bacteria Using an Antimicrobial Peptide from Class Ha Bacteriocins. Analytical Chemistry, 2014. 86(3): p. 1693-1700) and nuclear magnetic resonance systems (Chung, H. J., et al., Ubiquitous Detection of Gram-Positive Bacteria with Bioorthogonal Magnetqfluorescent Nanoparticles. ACS Nano, 2011. 5(11): p. 8834-8841; and Lee, H., et al., Chip—NMR biosensor for detection and molecular analysis of cells. Nature medicine, 2008. 14(8): p. 869-874).

The most commonly used method for gram-negative bacteria and endotoxin detection is the Limulus Amebocyte Lysate (LAL) testing that was approved by the FDA in 1983 (Seiter, J. A. and J. M. Jay, Comparison of direct serial dilution and most-probable-number methods for determining endotoxins in meats by the Limulus amoebocyte lysate test. Applied and Environmental Microbiology, 1980. 40(1): p. 177-178). This is one of the very few homogenous and instantaneous microbial tests that can provide results within minutes by interacting with the bacterial lipopolysaccharides. However, the test can generate false positives due to a lack of specificity and possible interferences with the sample components and other fungal lipopolyscharides (Elfin, R. J. and S. M. Wolff, Nonspecificity of the Limulus Amebocyte Lysate Test: Positive Reactions with Polynucleotides and Proteins. Journal of Infectious Diseases, 1973. 128(3): p. 349-352). This is why LAL testing is mostly used for quality control in drug manufacturing. Other developed methods include filtration and staining (Yazdankhah, S. P., et al., Rapid Method for Detection of Gram-Positive and Negative Bacteria in Milk from Cows with Moderate or Severe Clinical Mastitis. Journal of Clinical Microbiology, 2001. 39(9): p. 3228-3233), fluorescence-based flow cytometry (Langerhuus, S. N., et al., Gram-typing of mastitis bacteria in milk samples using flow cytometry. Journal of Dairy Science. 96(1): p. 267-277), and other biosensors based on the detection of lipopolysaccharide at the surface of the gram-negative bacteria (Chan, S., et al., Identification of Gram Negative Bacteria Using Nanoscale Silicon Microcavities. Journal of the American Chemical Society, 2001. 123(47): p. 11797-11798; and Liang, P.-S., T. S. Park, and J.-Y. Yoon, Rapid and reagentless detection of microbial contamination within meat utilizing a smartphone-based biosensor. Scientific Reports, 2014. 4: p. 5953).

Disclosed methods can be used for various purposes. In some embodiments, disclosed methods can be used to analyze a microorganism. The term “analyze” in reference to a microorganism, as used herein can refer to separating it from a solution, concentrating it in a solution or otherwise, manipulating it, detecting the presence thereof in a sample, affecting its properties, or some combination thereof. In some embodiments, disclosed methods can be utilized to detect one or more microorganisms in a composition, solution or sample. In some embodiments, disclosed methods can be utilized to identify one or more microorganisms, the class of a microorganism present, or some combination thereof in a composition, solution or sample. In some embodiments, disclosed methods can be utilized to quantify one or more microorganisms in a composition, solution or sample. In some embodiments, disclosed methods can be utilized to concentrate one or more microorganisms in a composition, solution or sample. In some embodiments, disclosed methods can be utilized to separate one or more microorganisms in a composition, solution or sample. In some embodiments, disclosed methods can be utilized to manipulate one more microorganisms in a composition, solution or sample. In some embodiments, disclosed methods can be utilized to detect, identify, quantify, concentrate, separate, manipulate, affect or any combination thereof one or more microorganisms in a composition, solution or sample.

Disclosed methods may provide many advantages when used for analytical purposes, such as detection, identification, quantification, concentration, separation, or any combination thereof. Some of which may include: microbial screening in less than 30 minutes; detection without the use of antibodies, aptamers or any biological receptor (receptor-free), which makes such methods less expensive and faster than any other available technology; and the methods can be used in solution (homogeneous assay), or can be adapted into a lateral flow assay (LFA).

As used herein “microorganism” will include bacteria (e.g., gram positive bacteria, gram negative bacteria and others), fungi (e.g., yeasts, molds and others), Archaea, protists (e.g. algae), viruses, any microscopic unicellular or multicellular organism or microscopic biological material (e.g., eukaryotic cells, and organelles), or combinations thereof.

Disclosed methods may be useful for “microbial screening”, which, as used herein means the detection, quantification, or some combination thereof of total microbial content (e.g., the total amount of microorganisms), detection, quantification or some combination thereof of specific microbial classes including detection, quantification or some combination thereof of the presence of fungi, bacteria, or viruses, or any combinations thereof (for example). In some embodiments disclosed methods can be utilized to identify, quantify or both, particular species or strains of microorganisms.

Microorganisms (e.g., bacteria, fungi and viruses) all include molecules on their surfaces. Some of these molecules are phylogenetic surface molecules or phylogenetic surface markers. Phylogenetic surface molecules are molecules that exist on a surface of a microorganism that do not necessarily elicit an immunogenic response by other organisms. Phylogenetic surface molecules include some molecules that are common across classes (e.g., there are some surface molecules that are present on all microorganisms) and some molecules that are only present in some types of microorganisms (e.g., gram positive bacteria have surface molecules that are not present in gram negative bacteria). Illustrative and non-limiting examples of phylogenetic surface markers include chitin in fungi, hydrophobin in filamentous fungi, lipopolysaccharides in gram-negative bacteria, and lipoteichoic acid in gram-positive bacteria. Targeting such markers can provide desired specificity for rapid microbial screening without the use of antibodies or other bioreceptors.

As used herein, “phylogenetic surface marker” or “PSM” includes phylogenetic surface markers and modified phylogenetic surface markers (e.g., a phylogenetic surface marker that has been reacted with some reagent(s)) which may include of one or more of the following: proteins, lipids, saccharide, in monomeric or polymeric form, or any combinations thereof.

As used herein, a “phylogenetic surface marker” or “PSM” refers to a surface molecule or biomolecule or a modified surface molecule or biomolecule that is present on the outer layer or surface of a microorganism and can be specific to the microbial type or taxonomic rank other than species. As used herein, a “probe” is a molecule, a biomolecule or a nanoparticle that is capable of generating or triggering a reaction to generate a signal including, but not limited to optical absorbance, fluorescence, luminescence or magnetic; the probe also interacts with a PSM on a microorganism. As used herein, a “receptor” includes molecules or biomolecules with recognition abilities (e.g., nucleic acids, aptamers, antibodies, enzymes or any other protein or molecule with recognition abilities).

Disclosed methods utilize probes to target one or more PSMs on one or more microorganisms for microbial screening or some other purpose. Disclosed herein are numerous illustrative probe and PSM combinations that can be utilized for microbial screening of one or more types of microorganisms in any type of sample.

Probes can include a number of types of materials including for example the following: metal beads, quantum dots and metal nanopoarticles (e.g., gold silver, copper, aluminum, etc); magnetic beads or magnetic nanoparticles; fluorophores, including but not limited to fluorescein derivatives, rhodamine, coumarin derivatives, green fluorescent proteins and combinations thereof, luminophores, including but not limited to luminol, transition metal complexes such as tris(bipyridine)ruthenium (II) chloride, or inorganic luminophores such as zinc sulfide or zinc orthosilicate; chromophores or chromogenic agents such as enzymes; electrogenic molecules; liposomes or vesicles; amine- or thiol-reactive probes; amine- or thiol-activated probes; and combinations thereof

Illustrative probes can include chemical groups that can form bonds with primary amines through acylation, alkylation, Mannich reactions and/or reductive amination, as well as other reactions. Illustrative amine-reactive probes can include isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, flurophenyl esters, formaldehyde and glutaraldehyde or other carbonyl (—CHO) reagents, or chemical groups requiring carbodiimides (such as EDC) to interact with amine groups. Illustrative thiol-reactive probes can include sulfhydryl-reactive chemical groups such as haloacetyls, maleimides, aziridines, acryloyls, arylating agents, vinylsulfones, pyridyl disulfides, TNB-thiols and other disulfide reducing agents, for example. Illustrative amine- or thiol-activated probes can include molecules that can become active (e.g., acquire fluorescent, luminescent, absorptive, magnetic or catalytic properties) following reaction with thiol groups. Specific examples of thiol-activated fluorophores can include, for example dibromobimanes (absorbance at 394 nm, emission at 490 nm) such as nBBr; diethylaminocoumarin (absorbance at 384 nm, emission at 470 nm) such as 7-diethylamino-3-(4′-maleimidylphenyl)-4-methylcoumarin (CPM); dimethylamonicoumarins (absorbance at 376 nm, emission at 465 nm) such as N-(7-dimethylamino-4-methylcoumarin-3-yl)maleimide (DACM); monobromobimanes such as mBBR and fluroscein-5maleimide. Specific examples of thiol-activated chromophores can include, for example thiol-activated enzymes (or disulfide bond-inhibited enzymes) such as enzyme-SSCH3 such as papin-SSCH3 that has DTNB (Ellman's reagent) as the chromogenic substrate and absorbs at 412 nm (EC=13,600 cm⁻¹M⁻¹); thiol-activated chromophores such as DTNB which absorbs at 412 nm (EC=13,600 cm⁻¹M⁻¹). Examples of amine-activated probes include aldehyde- or ketone-substituted diarylethenes.

An illustrative example of a probes and PSM combination includes the use of nanoparticles. In such an example, microorganisms can be coated with nanoparticles (e.g., the nanoparticles are the probe or include the probe) via interaction (e.g., some type of bonding) of PSMs on the microorganism with the nanoparticles. The result of the interaction is a coated microorganism. A coated microorganism is one which is at least partially coated with nanoparticles (in this example) that are interacting with one or more PSMs on the microorganism, stated another way, a coated microorganism is one in which at least some of the PSMs are interacting with nanoparticles as probes. The portion of the nanoparticle that interacts with the PSM can be referred to as the interactive portion of the nanoparticle or the targeting portion of the nanoparticle.

In some embodiments, disclosed methods utilize nanoparticles. Any type of nanoparticles (e.g., particles that are generally between 1 and 100 nanometers or 1 to 500 nm in size) can be utilized herein. The particular type of nanoparticle chosen for a particular type of method may depend at least in part on the particular microorganism(s) being targeted, the particular PSM being targeted, the particular purpose of the method, other factors, or any combination thereof. Options for nanoparticles are not limited and can vary greatly depending on the above noted factors. Nanoparticles can include one or more than one type or kind of material.

Nanoparticles for use herein can include, in addition to an interactive portion, at least one active material. Active materials can include materials having certain properties, materials that can be made to have certain properties, or materials that can be detected, monitored or modulated because of some property. Illustrative types of active materials can include plasmonic materials, materials with optical properties or materials that can be detected or monitored using one or more wavelengths of energy, materials that are photothermal or have photothermal properties, materials that can be detected, monitored or modulated based on their mechanical properties, materials that can be detected, monitored or modulated based on their electrical properties, materials can be detected, monitored or modulated based on their magnetic properties, or any combination thereof. Nanoparticles can include one or more than one active material, can include a single material that has more than one type of active material property (e.g., a single material can be both an optical active material and a magnetic active material), or any combination thereof. In some embodiments, nanoparticles can include both an interactive portion and an active material. In some embodiments, a first material or portion of the nanoparticles can function as an interactive portion and a second material or portion of the nanoparticles can function as an active material. In some embodiments, the same material or portion of the nanoparticle can function both as an interactive portion and an active material.

In some embodiments, useful nanoparticles can include active materials that have plasmonic properties such as plasmonic elements or plasmonic materials. Illustrative plasmonic materials can include, but are not limited to gold (Au), silver (Ag), copper (Cu), rhodium (Rh), aluminum (Al), platinum (Pt), palladium (Pd), Nickel (Ni), zinc oxide (ZnO), indium-tin-oxide (ITO), or combinations thereof. In some embodiments, the nanoparticles can include nanoparticles that include gold (Au). Microorganisms coated with plasmonic containing nanoparticles can be referred to herein as having a plasmonic cell nanocoating. As the nanoparticles aggregate on the surface of the microorganism, the color of the solution containing the coated microorganisms or the coated microorganisms themselves will change because of the plasmonic nature of the plasmonic material. For example, in a specific embodiment where the nanoparticles include gold, aggregation of nanoparticles on the surface of the microorganisms will cause a color shift of a solution (for example) from red to dark blue.

An illustrative example of a targeting portion of a nanoparticle includes hydroxyapatite. Hydroxyapatite containing nanoparticles can be useful to target gram positive bacteria because gram positive bacteria include lipoteichoic acid as a PSM and hydroxyapatite interacts specifically with lipoteichoic acid. Alternatively, hydroxyapatite containing nanoparticles can be useful to target gram positive bacteria because they have teichoic acid as a PSM and hydroxyapatite interacts specifically with teichoic acid. The hydroxyapatite forms a nanoshell around the gram positive bacteria.

In some embodiments, a nanoparticle could include a plasmonic material as an active material and a targeting portion. In some embodiments, the second material could be hydroxyapatite, for example. The nanoparticle could bind to the lipoteichoic acid as the PSM on gram positive bacteria through the hydroxyapetite and the plasmonic part, for example the gold part of the nanoparticle, could change the color of the solution upon aggregation of the nanoparticles.

The PSM being targeted by the nanoparticle may be a phylogenetic surface molecule or a modified phylogenetic surface molecule. Modified phylogenetic surface molecules can be formed by reacting one or more reagents with the microorganism in order to chemically modify the PSM, this modified PSM can be referred to as an active PSM. Modifications that can be made to PSMs can vary greatly, depending on a number of factors, including but not limited to the identity of the PSM, the type of probe being utilized, the purpose of the method, other factors, or any combination thereof. Illustrative methods of activating one or more PSMs can include, for example reduction and deacetylation. For example, disulfide bridge containing (Dsbc) proteins can be activated by reducing the Dsbc proteins using a reducing agent or through electrochemical reduction; or chitin can be deacetylated. FIG. 13 schematically illustrates reduction of Dsbc proteins on microorganisms followed by combination with a probe to form a coated microorganism. FIG. 13 specifically shows the general concept of cell nanocoating with probes (nanomaterials including nanoparticles, molecules and biomolecules), after activation of the microbial cells by reduction (using TCEP for instance) or deacetylation (using NaOH for instance). Cell nanocoating can be performed for different purposes.

In specific illustrative embodiments designed to detect, identify, quantify, concentrate, separate, manipulate, affect or any combination thereof all microorganisms (e.g., all bacteria, fungi and viruses) in a sample, a phylogenetic surface molecule of interest can include disulfide bridges in protein molecules on the surface. All microorganisms include protein molecules having disulfide bridges. This particular PSM can be modified by reducing the disulfide bridges to form thiol groups. The free thiol groups can then interact specifically with gold containing nanoparticles as a probe to form a gold nanoparticle layer on the surface of the microorganism. In such embodiments, gold can act both as the targeting portion of the probe and the active material of the probe.

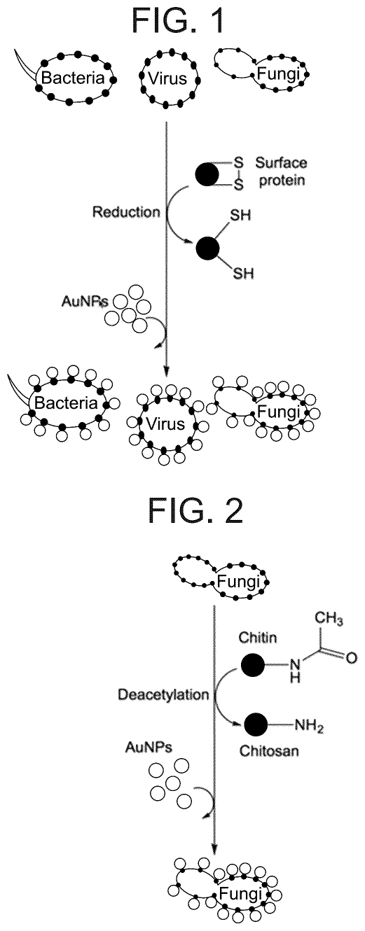

In some embodiments, the disulfide bridges can be reduced before the gold nanoparticles are added, at substantially the same time as the gold nanoparticles are added, or some combination thereof. Reduction of the disulfide bridges to form free thiol groups can be accomplished using known reducing agents. Illustrative examples of useful reducing agents can include, for example thiols such as 2-mercaptoethanol (BME), 2-mercaptoethylamine-HCl (2-MEA-HCl), cysteine-HCl, dithiothreitol (DTT), zinc (Zn), glutathione, acids or combinations thereof. Additional illustrative examples of useful reducing agents can include, for example tris(2-carboxyethyl)phosphine (TCEP), tris(2-carboxyethyl)phosphine hydrochloride (TCEP-HCl), or combinations thereof. Reduction of the disulfide bridges to form a PSM and combination thereof with gold nanoparticles is illustrated schematically by FIG. 1.

In specific illustrative embodiments designed to detect, identify, quantify, concentrate, separate, manipulate, affect or any combination thereof fungi, a PSM of interest can include chitin. All fungi include chitin on the surface. This particular PSM can be modified by deacetylating the chitin and converting it from an amide to a free amine group or more specifically to chitosan. The free amine groups can then interact specifically with gold containing nanoparticles to form a gold nanoparticle layer on the surface of the microorganism. In such embodiments, gold can act both as the targeting portion of the nanoparticle and the active material of the nanoparticle. FIG. 13 illustrates deacetylation of chitin on fungi followed by combination with a probe. In some embodiments, the amide groups can be deacetylated before the gold nanoparticles (for example) are added, at substantially the same time as the gold nanoparticles are added, or some combination thereof. Deacetylation of the amide groups in chitin to form free amine groups can be accomplished using known deacetylating agents. Illustrative examples of useful deacetylating agents can include for example strong bases, enzymes, or other chemicals. Illustrative specific examples of useful strong bases can include but are not limited to lithium hydroxide (LiOH), sodium hydroxide (NaOH), potassium hydroxide (KOH), magnesium hydroxide (Mg(OH)₂), calcium hydroxide (Ca(OH)₂), or combinations thereof for example. Illustrative specific examples of useful enzymes include but are not limited to deacetylase. Illustrative specific examples of useful other chemicals include but are not limited to acetic anhydride. Deacetylation of the chitin to form a PSM and combination thereof with gold nanoparticles is schematically depicted in FIG. 2.

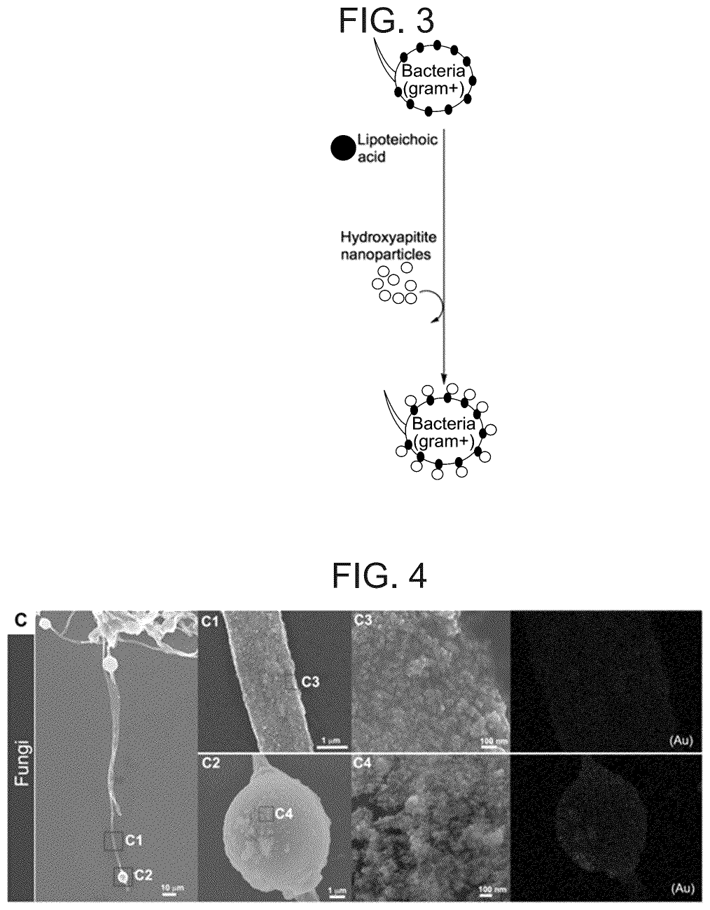

In specific illustrative embodiments designed to detect, identify, quantify, concentrate, separate, manipulate, or any combination thereof gram positive bacteria, a PSM of interest can include lipoteichoic acid. All gram positive bacteria include lipoteichoic acid on the surface. Lipoteichoic acid, without any modification interacts specifically with hydroxyapatite containing nanoparticles. The hydroxyapatite containing nanoparticles form a nanoshell around the gram positive bacteria. Combination of the hydroxyapatite containing nanoparticles with the gram positive bacteria containing lipoteichoic acid as a PSM is schematically depicted in FIG. 3. In such an embodiment, the hydroxyapatite acts at least as the targeting portion of the probe.

In specific illustrative embodiments designed to detect, identify, quantify, concentrate, separate, manipulate, affect or any combination thereof gram negative bacteria, a PSM of interest can include lipopolysaccharides. All gram negative bacteria include lipopolysaccharides on the surface. Lipopolysaccharides, with or without any modification may interact specifically with nanoparticles that contain organic or inorganic materials with specific interactions with the lipopolysaccharides, and act as the targeting portion of the probe.

In specific illustrative embodiments a probe that chemically or biologically interacts with the target, e.g., one or more microorganisms can also be utilized. The probe can emit a signal (e.g., fluorescence, luminescence, absorbance, etc.), perform a function on the microorganism, or some combination thereof.

In some embodiments, nanoparticles can also include a magnetic material or component as the active material. Nanoparticles that contain magnetic materials may be useful for enabling or increasing the ease of detecting, identifying, quantifying, concentrating, separating, manipulating, affecting or any combination thereof one or more microorganisms. Magnetic materials can also be combined with any other type of material (e.g. any type of targeting portion) to form a nanoparticle. In some embodiments, the inclusion of magnetic materials in nanoparticles can be useful for enabling concentration, separation, or combinations thereof. After the nanoparticles are combined with the microorganisms and coat the microorganism, the coated microorganisms will then be magnetic and can be controlled using an external magnet, magnetic field, or both. Illustrative magnetic materials can include, for example iron, iron oxide, magnetite, or combinations thereof as well as any other material which has or can be made to have magnetic properties.

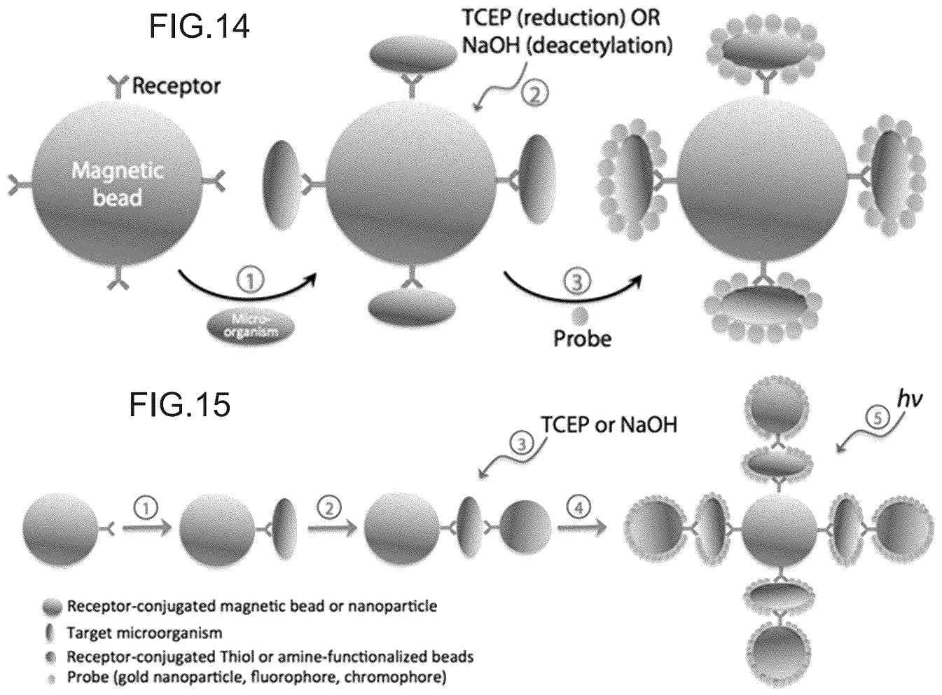

In some embodiments, magnetic materials including a receptor can be utilized to separate a specific microorganism from others as well as the overall sample. In such embodiments, magnetic beads (for example) including a receptor (e.g., antibody, DNA, aptamers, proteins, etc.) can be combined with a sample containing the microorganism. The microorganism will then be coupled to the magnetic bead via the receptor. A PSM on the microorganism can then be activated via reduction or deacetylation after which a probe can be combined. This is schematically illustrated in FIG. 14. FIG. 14 shows the application of cell nanocoating for microbial identification. Step (1) shows detection of target microorganism using beads (magnetic, silica or other materials) conjugated to a specific receptor (antibody, DNA, aptamers, protein). Step (2) shows activation of the microorganisms by reduction or decetylation. Step (3) shows addition of probes (nanoparticles, molecules, biomolecules) capable of interacting with the thiol or amine groups at the microbial surface. The magnetic beads in this example are used for separation purposes, if the separation is not necessary, other beads could alternatively be used such as silica, polysterene, or any other organic or inorganic materials or a combination of the two, or no beads could be utilized.

In some disclosed embodiments, magnetic nanoparticle coated microorganisms can be used to separate the microorganisms from other components in the sample, solution or composition. For example, a solution containing the magnetic nanoparticle coated microorganisms could be placed in a container of some sort, an external magnet can be used to attract the magnetic nanoparticle coated microorganisms and then at least some of the remaining components of the solution can be removed. This can enable separation of microorganisms (e.g., bacteria, fungi, viruses, or any combination thereof) via the use of disclosed methods. Such techniques could also be utilized to concentrate the microorganisms by separating (e.g., using a method such as that discussed above) them, adding additional solution and repeating the separation steps. Such separation methods, concentration methods, or combinations thereof can be accomplished using any useful devices, including, for example lateral flow assay devices.

In some embodiments, more than one type of probe can be utilized in a method. For example, a second probe can be utilized in disclosed methods. In some such methods, the second probe can afford a method of detection, another way of modulating the microorganisms, another way of separating, identifying, or otherwise, one microorganism from another, or any combination thereof. Specific examples of such methods can include methods where the first probe includes hydroxyapatite as the targeting portion and a magnetic material as the active material. The hydroxyapatite could be specific for gram positive bacteria because it interacts with lipoteichoic acid as a PSM, specific for gram negative bacteria because it interacts with a modified lipopolysaccharide as a PSM, or inclusive of both gram positive and gram negative bacteria because it interacts with both lipoteichoic acid and modified lipopolysaccharides. Because the probe is also magnetic, the chosen microorganism (e.g., gram positive bacteria, gram negative bacteria, or both) can be separated using an external magnet or magnetic field. Once the magnetic coated microorganisms are separated from the remaining components of the solution or sample, the disulfide bridges present in the external proteins (PSM) can be reduced and then combined with a second probe, for example a gold containing nanoparticle (that acts as both the targeting and the active portions) which will change color when aggregation occurs in order to detect the desired microorganism(s). Use of a plasmonic containing nanoparticle (e.g., gold containing nanoparticles) to interact with the disulfide bridges (after being reduced) that are present on the surfaces of all microorganisms, after the microorganism of interest has been separated from the remainder of the sample or solution (using a first probe that contains magnetic particles) affords the ability to detect virtually any type of microorganism even if the PSM used for separation does not easily afford detection. In some embodiments, the first probe could be at least partially removed from the microorganism and in some embodiments, the first probe can remain and the second can simply be added in addition to the first.

Disclosed methods that include detection of one or more microorganisms can include detecting virtually any property or a change in a property due to the presence of a coated microorganism. Illustrative properties can include but are not limited to optical properties such as visual or colorimetry changes such as a change in color, or spectroscopic changes; electrochemical properties such as a change in electrical signal; magnetic properties such as a change in relaxation time in nuclear magnetic resonance spectroscopy; photothermal properties; mechanical properties; or any combinations thereof.

Disclosed methods can also be utilized to quantify the amount of a microorganism in a sample. In some embodiments where a plasmonic material is utilized as an active material in a nanoparticle, the amount of targeted microorganism can be determined by visual inspection of the color change, spectroscopic inspection of the color change (e.g., using UV spectroscopy and correlating color change with concentration using standards), or some combination thereof. Probe materials other than plasmonic materials could utilize other types of methods for quantification of the targeted microorganisms which would be dependent on the particular active material.

Disclosed methods can also be utilized to identify the class of microorganism, the sub class of microorganisms, the particular identity of the microorganisms or some combination thereof. In some embodiments this can be accomplished by using disclosed methods to separate and capture the microorganisms in a sample or solution and then using known methods of identifying (e.g., culturing the microorganisms, PCR based methods, microarray based methods, etc.) the particular genus, species, strain, or a combination thereof. In some embodiments this can be accomplished by using disclosed methods to separate and capture the microorganisms in a sample or solution, then utilizing a PSM for a particular subgroup within the larger group that was first captured and separating and capturing that subgroup, and repeating the process until the level of specificity is obtained in the identification. Because every species has both different and shared phylogenetic surface markers, a choice of a second PSM can be utilized to obtain a smaller subset than what was originally captured.

FIG. 15 illustrates microbial identification using cell nanocoating an optional signal amplification system, or cell nanocoating sandwich assay. Step (1) shows microbial capture with a bead conjugated with a specific receptor. The beads or nanoparticles can be magnetic if sample separation is needed or if the detection is performed with nuclear magnetic resonance techniques. Step (2) shows a sandwich reaction: the complex formed from step (1) interacts with the second bead conjugated with the same receptor and with thiol or amine groups. Step (3) shows microbial activation: the complex formed from step 1 and 2 is exposed to TCEP for reduction or NaOH for deacetylation for example. Step (4) shows the addition of the probe (nanoparticle, molecule), which results in cell nanocoating. Step (5) shows that the nanocoated complex can be detected using absorption spectrometry, colorimetry, fluorescence, luminescence or electrochemical techniques, for example.

Disclosed methods can also utilize various processes for sample processing and preparation including as a specific example only (and not in any way limiting) the processes disclosed in PCT Application Number PCT/US2016/019772, filed on Feb. 26, 2016, entitled “DETECTION ASSAYS AND METHODS”, the disclosure of which is incorporated herein by reference thereto.

Disclosed methods can be accomplished in solution, in some type of assay, by using some particular device (e.g., some assay type of device, or colorimetric device), or any combination thereof. In some embodiments, disclosed methods can be accomplished in an assay, for example a homogeneous assay, an inhomogeneous assay, a lateral flow assay, or other types of assays. The present disclosure is illustrated by the following examples. It is to be understood that the particular examples, assumptions and procedures are to be interpreted broadly in accordance with the scope and spirit of the disclosure as set forth herein.

### Examples

Materials

Gold (III) chloride trihydrate, trisodium citrate dehydrate, trisodium citrate dehydrate, tris(2-carboxyethyl)phosphine hydrochloride (TCEP), sodium Hydroxide (NaOH), rhodamine 6G, and 2-mercaptoethanol (BME) were purchased from Sigma-Aldrich (St. Louis, MO, USA). All media for microbial cultures were also purchased from Sigma-Aldrich (St. Louis, MO, USA). All reagents were used as received unless otherwise specified. All microorganisms were purchased from the American Type Culture Collection (ATCC).

Preparation of Gold Nanoparticles (AuNP) Gold nanoparticles were prepared based on a modification of Turkevich's methods (Bui, M.-P.

N.; Ahmed, S.; Abbas, A. Nano Letters 2015, 15, 6239-6246; Turkevich, J.; Stevenson, P. C.; Hillier, J. Discuss. Faraday Soc. 1951, 11, 55-75; and Grabar, K. C.; Freeman, R. G.; Hommer, M. B.; Natan, M. J. Anal. Chem. 1995, 67, 735-743, the disclosures of which are incorporated herein by reference thereto). Specifically, 1 mM of HAuCL4 solution was boiled on a hot plate for 5 min, followed by adding 10 mL of briefly preheated 38.8 mM sodium citrate solution. After 30 s stirring, the solution was taken off from the hot plate and cooled to room temperature. The final solution has ruby red color with a strong absorption peak at 520 nm as measured by a UV-vis spectrophotometer. The size of the AuNPs was characterized as 12±2 nm in diameter using transmission electron microscopy (TEM, FEI Technai T12).

Preparation of the Bacteria Suspensions Escherichia coli Castellani and Chalmers (ATCC 25922) were grown on tryptic soy agar (TSA) and Lactobacillus delhrueckii subsp. Bulgaricus were grown on the MRS Agar at 37° C. overnight and store at 4° C. until use. Before use, the concentration of the microbial suspension was evaluated by measuring the optical density, and a serial of ten-fold dilution was performed to prepare different microbial concentration from 10 to 10⁸ cfu.mL⁻¹. The microbial concentrations were confirmed using the BD Accuri™ C61 flow cytometer (BD Biosciences, USA), hematocytomer and plate counting methods.

Preparation of the Fungi Suspensions

Saccharomyces cerevisiae var. boulardii (ATCC MYA-796 ™) were grown in the YM broth at 30° C. with 200 rpm shaking overnight. Mucor circinelloides (ATCC® MYA-3787™) were grown in the Potato dextrose agar (PDA) media at 25° C. All samples were then centrifuged twice for 5 minutes at 12,000 rpm and suspended in water. The microbial cultures were stored at 4° C. and reactivated at growth temperature before use. For deacetylation, 50% (w/v) NaOH solution was added to samples at equal volume ratio. The mixture was vortexed well and rotated at 30 rpm on tube revolver for 25 minutes. Then, the solution was centrifuged at 10,000 rpm for 5 minutes and re-suspended in nanopure water. The solution pH was further adjusted to 7.0 using 1M HCl solution. The microbial concentration was evaluated and different dilutions were prepared. The microbial concentrations were confirmed using the BD Accuri™ C61 flow cytometer (BD Biosciences, USA), hematocytomer and plate counting methods.

### Example 1: Detection of Fungi Using Chitin as a PSM

In this example chitin, which is a phylogenetic surface molecule on fungi was converted into chitosan, which provides free reactive anime groups, for subsequent interaction with gold nanoparticles, where the gold nanoparticles function as both the targeting portion and the active portion.

Deacetylation of Chitin. First, the sample matrix (e.g., food matrix) of a sample that contained fungi (ex. Mucor) was removed using a separation technique to obtain a supernatant. Then, the pellet was suspended in nanopure water. Next, the solution was centrifuged once to remove the nanopure water (10 k, 5 min). Then, a volume of a 50% NaOH solution equal to the volume of the sample was added to the fungi sample and heat was generated thereby. The solution was then vortexed and subsequently mixed for about 20 to 30 minutes on a rotator, and then vortexed again. Then, the solution was centrifuged at 10 k for about 5 min and this was repeated until the pH of the solution was around 6.5. Then water was added to disperse the fungi, sonicated and check optical density (OD) value to estimate concentration, using a UV visible spectrometer.

Combination with Nanoparticles in Solution. The deacetylated fungi were then mixed with gold nanoparticles (AuNPs) prepared as above in equal volume and then the mixture was vortexed. The solution changed color from red to purple or dark blue. FIG. 4 shows scanning electron microscopy (SEM) images at various scales (10 μm, 1 μm, and 100 nm) and corresponding energy dispersive X-ray spectroscopy (EDX) maps of the nanocoated fungi which shows gold (Au). Alternatively, the nanoparticle coated fungi could be deposited on a paper strip of a lateral flow assay (LFA). A dark dot or band would appear once the nanoparticle coated fungi traveled into the absorbent pad of the paper strip.

Combination with Nanoparticles on a Membrane. One (1) mL of the deacetylated fungi were injected through an extruder containing a nitrocellulose or other filtration membranes with a pore size smaller than 500 nanometers. The fungi concentrated in the center of the membrane. After waiting until the membrane dried, it was dipped into a gold nanoparticle solution for about 1 min. The membrane changed color from white to blue showing the presence of the nanoparticle coated fungi.

### Example 2: Detection of Total Microorganisms Using Disulfide Bridges in Proteins as PSM

Methods such as these can be performed in solution, on a lateral flow assay or on a filtration membrane for example. All such methods start by reducing the sample. In this step a reducing agent (such as beta-mercaptoethanol (BME)), concentration needs to be higher than 150 mM) or tris(2-carboxyethyl)phosphine (TCEP)) is used to reduce the disulfide bridges into free reactive thiol groups on the surface of microorganisms.

Reduction of the Sample. 1 mL samples at different diluted concentrations (10 fold dilution) of E. coli Chastellani and Chelmer in nanopure water or Mucor circinelloides in milk ( 1/1000 dilution) were placed in Eppendorf tubes. A control with 1 mL of Nanopure water and 1 mL of diluted milk was also ran both without and without the reducing agent (BME). The concentration of beta-mercaptoethanol (BME) in the water samples was 298 mM and the concentration of the BME in the milk samples was 290 mM (alternatively, 200 μL of 10 mM TCEP solution could be added to each tube to reduce the disulfide bridges). The tubes were incubated for about 10 minutes at room temperature.

Combination with Nanoparticles. The gold nanoparticles function as both the targeting portion and the active portion. The samples were mixed with 200 μL to 600 μL AuNP prepared as above. The gold nanoparticles were synthesized according to the procedure described in: Bui, M-P.; S, Ahmed; Abbas, A. “Single Digit Pathogen and Attomolar Detection with the Naked Eye using Liposome-Amplified Plasmonic Immunoassay”, Nano Letters, 2015,_15 (9), 6239-6246.

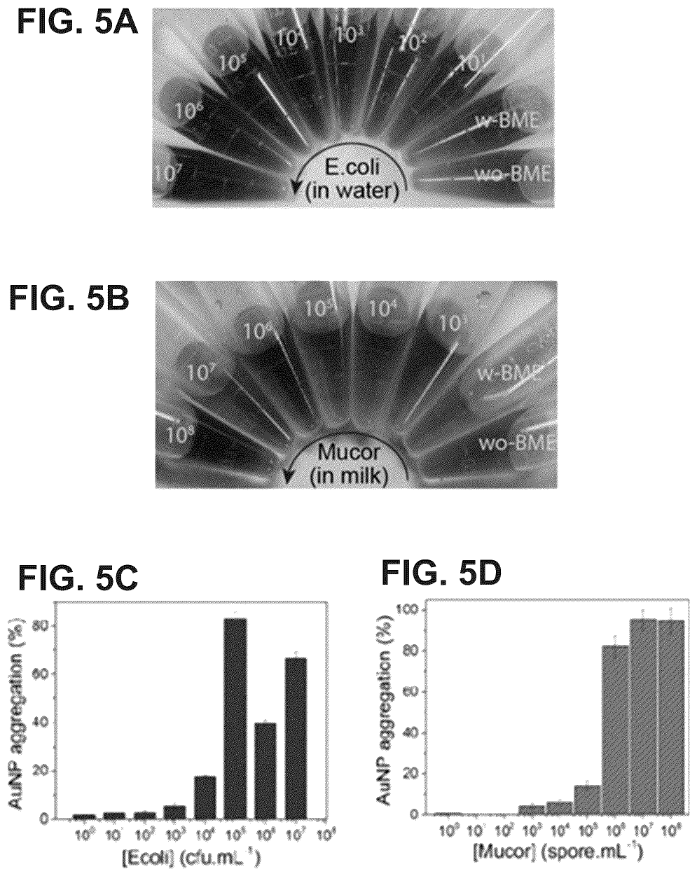

Visual Detection. FIG. 5A shows a photograph and FIG. 5C shows the nanoparticle aggregation percentage versus the E. coli concentration (cfu/mL) for the samples of E. coli in water and FIG. 5B shows a photograph and FIG. 5D shows the nanoparticle aggregation percentage versus the E. coli concentration (cfu/mL) for the samples of E. coli in milk.

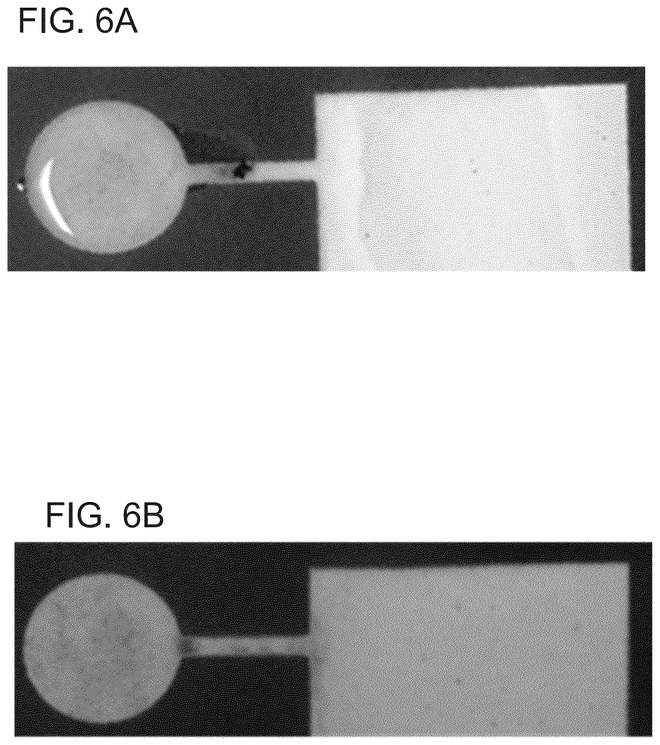

Detection in a lateral flow assay: FIG. 6A shows an image of a control (with no microorganisms) and FIG. 6B shows an image of a sample containing bacteria. The dark spot shows the aggregation of gold nanoparticles on the bacterial surface in the sample that includes the bacteria. The test was performed in less than 10 minutes. First the bacteria were reduced using BME as illustrated above and then added to the paper strip, followed by the addition of a gold nanoparticle solution.

Detection by extrusion or filtration. The solution containing the disulfide bridges reduced with BME or TCEP can be extruded through a nitrocellulose membrane 1 time (for example), using an extruder. Then, the solution can be removed using the extrusion kit. The membrane can then be dried in the air at room temperature, for example for about 2 to 3 min. The membrane can then be immersed in the gold nanoparticle solution for about 1 minute. Then, the membrane can be taken out and air dried for about 2 to 3 minutes. The color change can be observed from inside the circular membrane. A color change from red to purple or blue would indicate the presence of microorganisms.

### Example 3: Detection of Fungi Using Lipoteichoic Acid as PSM

This example is based on the specific interaction of a 1% hydroxyapatite nanoparticle solution (HAP-NP) prepared form a HAP nanopowder (<200 nm size) acquired from Sigma Aldrich with lipoteichoic acid present on the surface of gram (+) bacteria. First, a 1% HAP-NP solution was adjusted to pH=7.0. Then, a 1 mL sample containing gram (+) bacteria (ex. Lactobacillus) was mixed with 200 μL HAP-NP (1%) for about 15 min. The solution is allowed to sit for about 10 to 30 min to allow HAP-NP aggregation on the surface of the bacteria. Alternatively, the nanoparticles including hydroxyapatite could also include magnetic material that could be used for separation purposes of the gram (+) bacteria. Alternatively, the nanoparticles could include both hydroxyapatite and a plasmonic material (e.g., gold) that could be used for detection purposes of the gram (+) bacteria.

### Example 4: Detection of Microorganisms Using Disulfide Bonds in Proteins as PSM

Filamentous fungi and spores are surrounded with a layer of hydrophobins; surface proteins that contain four disulfide bonds. Mixing a fungal suspension with a reducing agent would reduce the disulfide bonds, yielding free reactive thiol groups. The subsequent addition of gold nanoparticles (AuNPs) would cause the nanoparticles to interact with the thiol groups and spontaneously form a thin monolayer coating on the fungal surface. At high microbial concentrations, the interaction results in visible color change of the suspension from red (single nanoparticles) to dark blue (nanoparticle assembly), caused by a plasmonic coupling of localized surface plasmon resonance in gold nanoparticles.

A 400 μL sample of a fungal suspension of Mucor (prepared as above with a final concentration of 10⁸ cfu.mL⁻¹) was mixed with 80 μL of a 10 mM Tris(2-carboxyethyl)phosphine (TCEP) solution and incubated for 5 min. Then, 400 μL, of AuNP solution was added to the mixture, and the absorbance of the sample at 600 nm was immediately measured using a UV-vis spectrometer.

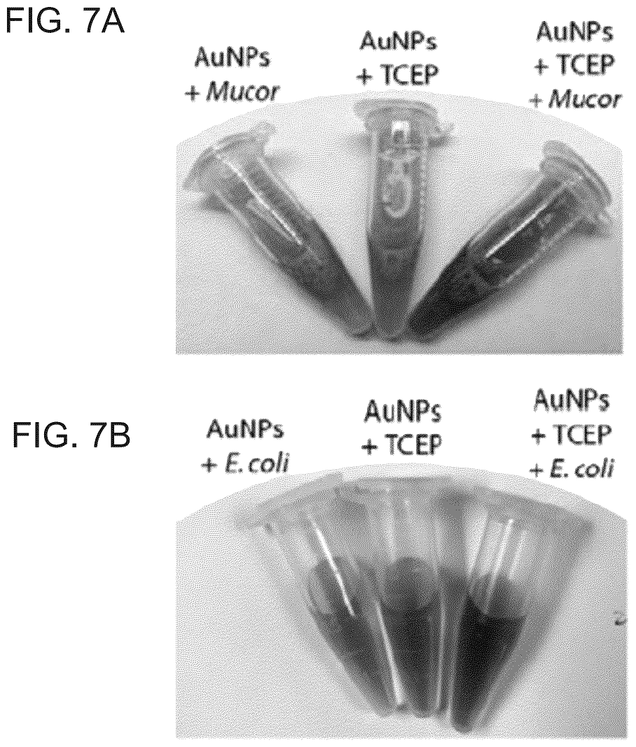

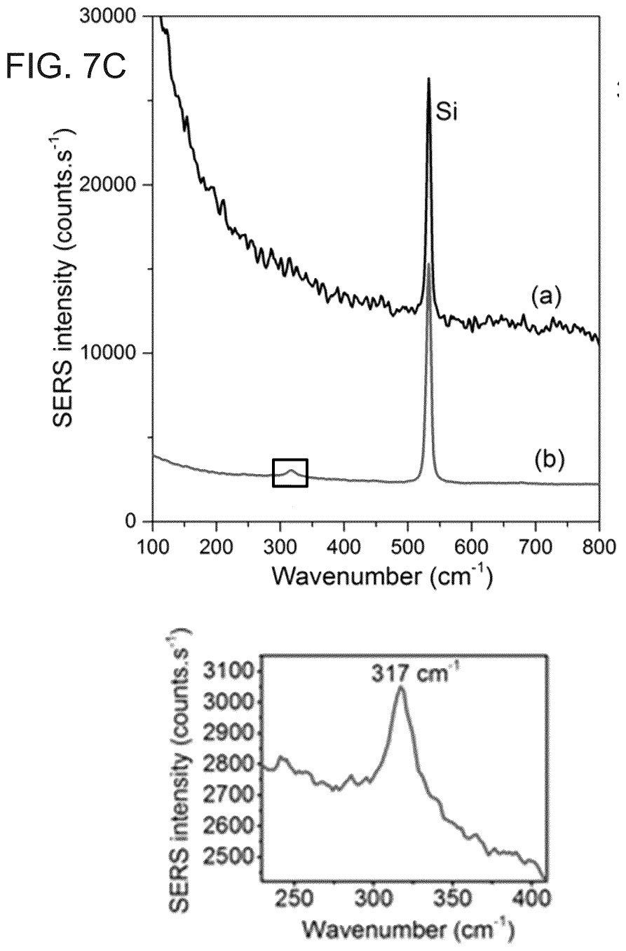

As expected, addition of TCEP to the fungal followed by the addition of AuNPs results in immediate color change from red to dark blue (FIG. 7A). Controls containing AuNPs and fungi or AuNPs with TCEP did not show any change. Replacing TCEP with another reducing agent such as 2-mercaptoethanol resulted in similar assembly (results not shown herein).

To assess the specificity of the screening for filamentous fungi, we decided to use, non-filamentous fungi Saccharomyces boulardii, gram-negative bacteria Escherichia coli, and gram-positive bacteria Lactobacillus delbrueckii subsp. Indgaricus as control samples since we assumed that these microorganisms do not contain hydrophobin or any other Dsbc protein layers. While gram-positive bacteria are surrounded with a peptidoglycan layer, the outer layer of gram-negative bacteria is composed of lipopolysaccharides and porins. According to the literature, porins are beta barrel proteins that do not exhibit any disulfide bonds. Both microbial types are sometimes encapsulated in a crystalline layer of proteins or glycoproteins called an S-layer. However, S-layer proteins are reported to very rarely contain sulfur-containing amino acids, if at all. Hence, the control microorganisms were not expected to generate any interaction. To our surprise, the same reaction and color change was also observed with the non-filamentous fungi and with both gram-negative and gram-positive bacteria (FIG. 7B). Whole genome sequencing of L. delhrueckii subspecies bulgaricus revealed the absence of S-layer protein gene, and S. boulardii does not contain hydrophobin. These results suggest that the Dsbc proteins probably form a different layer or distribution on the microbial surface, or that Dsbc proteins are present in previously reported surface layers but have never been identified. Twenty species of bacteria, yeast and mold (Yeasts: Saccharomyces boulardii, Torulaspora delbrueckii, Rhodotorula mucilaginosa, Cryptococcus carnescens, Candida kefry; Molds: Penicillium commune, Aspergillus niger, Cladosporium spp., Penicillium roquefurti, Radopholus simihs; Dimorphic fungi: Mucor circine ilDides; Gram-positive bacteria: Lactobaeihts de/hrr.reckii subsp. btilgarictis, 1′Wihicillirr-resistant S Phylococcus aureus (MRSA), Listeria mortocylogenes; and Gram-negative bacteria: Escherichia Coli, Salmonella) were tested and all showed a positive reaction with gold nanoparticles after reduction with TCEP. Surface-enhanced Raman analysis of the coated microorganisms revealed a peak at 317 cm⁻¹ assigned to Au—S bonds, thus confirming the existence of thiol-containing molecules on the microbial surface (FIG. 7C). As for viruses, the capsid is usually a repetition of one or two proteins. A simple search and analysis on the Protein Data Bank (PDB) or published literature indicate the presence and importance of disulfide bond-containing proteins in viral capsids, suggesting a universal aspect of Dsbc surface proteins or glycoproteins in microorganisms, and the potential of using them as markers for total microbial load. The existence of the Dsbc protein layers could be largely explained by the fact that disulfide bonds confer an evolutionary advantage to microorganisms by stabilizing and strengthening their cell walls exposed to fluctuating or extreme environmental conditions.

For macroscopic organisms such as animal and plant tissues, disulfide bonds are generally present in lysosomal proteins, secretory proteins, and in some membrane proteins. However, their presence does not seem to be ubiquitous in a way that can cause assembly with short-range plasmonic coupling. In fact, the plasmonic coupling that causes a change in color vanishes exponentially with the increasing distance between the nanoparticles, and becomes weak or inexistent beyond 20 nm distance as we have previously reported.

### Example 5: Detection of Fungi Using Chitin as PSM

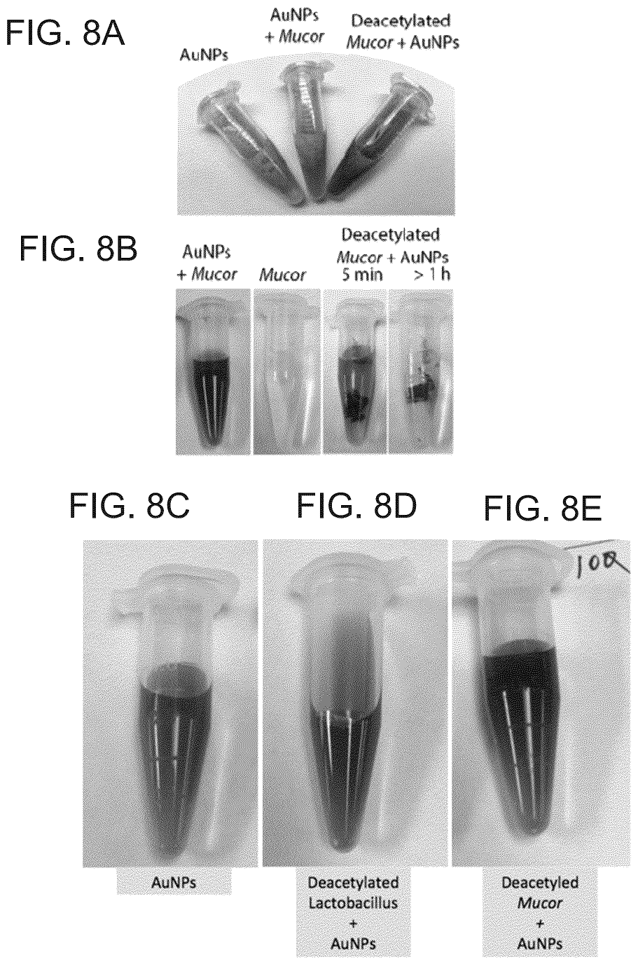

In addition to total microbial load, it may be useful in rapid screening to know the microbial type present in the sample. This can be achieved by taking fungi as an example. To enable specific detection of fungi in a microbial suspension, chitin, a rigid polysaccharide-based three-dimensional network, unique to fungal cell walls and the exoskeletons of arthropods was used as a PSM. Similar to the disulfide bonds, chitin requires activation to enable its interaction with gold nanoparticles as a probe. The activation is obtained by converting chitin into chitosan through a deacetylation process by adding a 50% sodium hydroxide solution for 30 min. The reaction yields free reactive primary amine groups at the fungal surface. The subsequent addition of an equal volume of AuNP solution to deacetylated fungi sample spontaneously results in very dense and highly stable cell nanocoating. A color shift from red to dark blue can be seen in the fungal suspension at high concentrations (FIGS. 8A and 8B). Such color shift is not seen when only the AuNPs are alone in solution (FIG. 8C) Although the cell wall in gram-negative bacteria contains N-Acetylglucosamine (a monomeric unit of the polymer chitin), the deacetylation of both gram-negative and gram-positive bacteria (FIG. 8D) does not yield any color change, indicating that the test is specific to fungi (FIG. 8E).

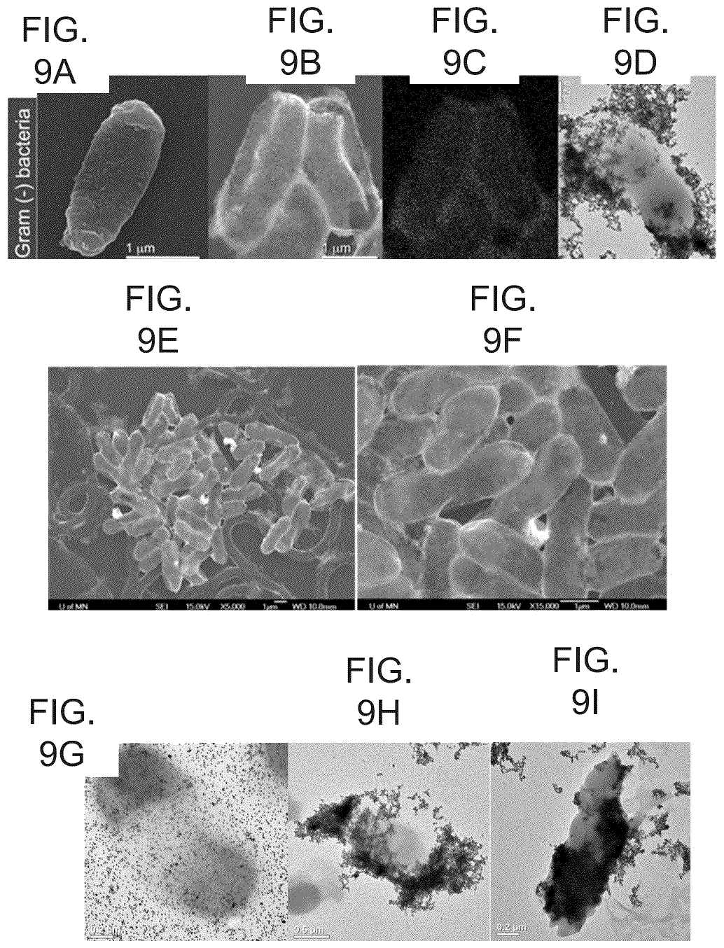

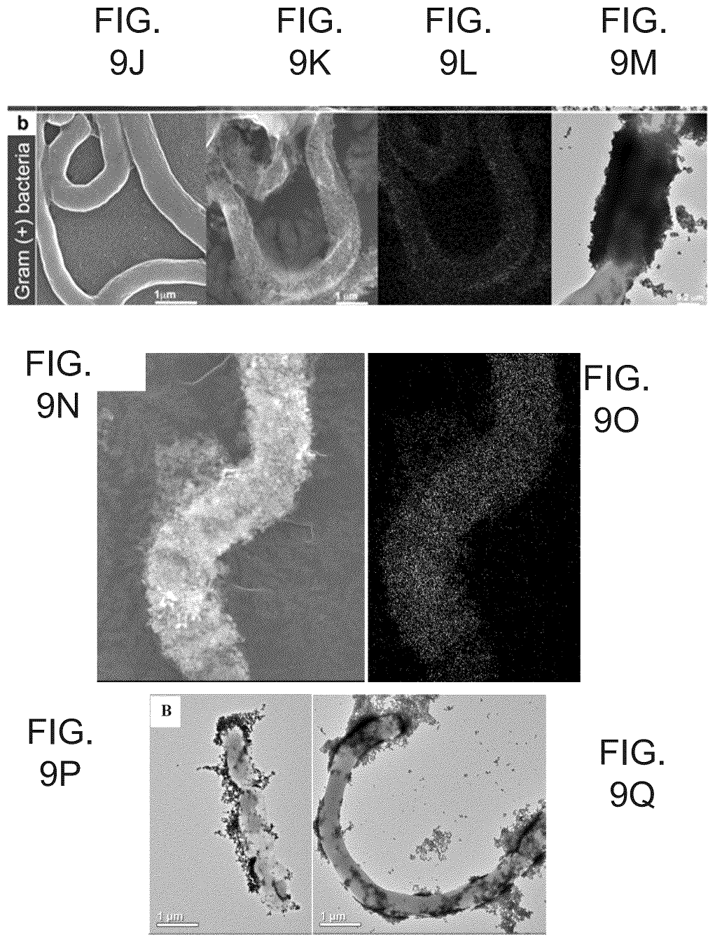

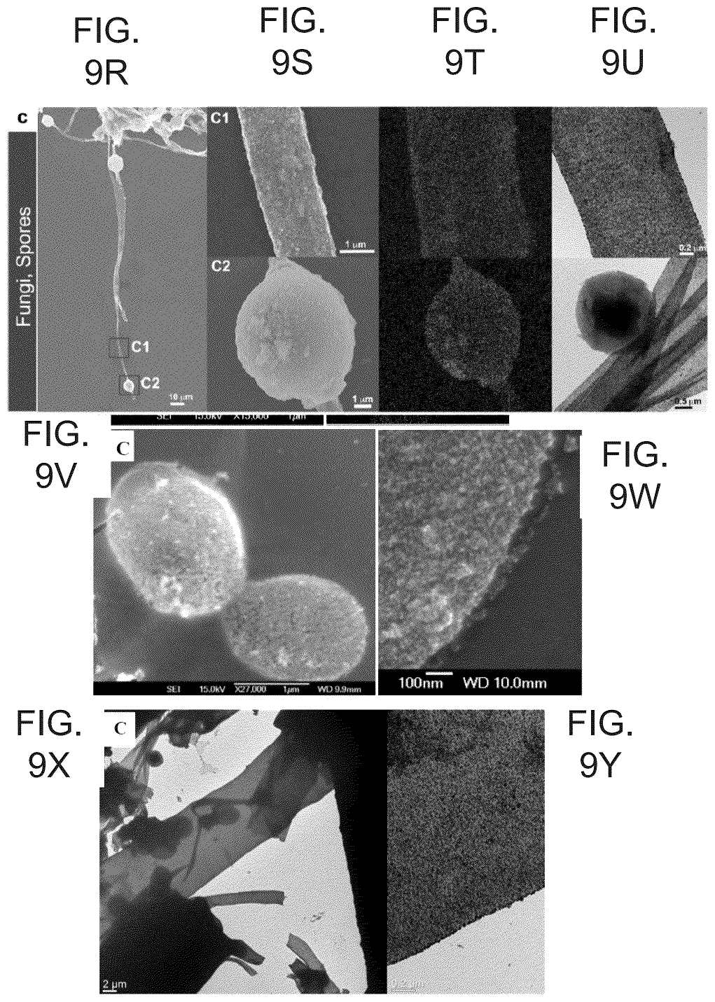

To confirm that the color shift (i.e nanoparticle assembly) in the microbial solutions is the result of cell nanocoating, bacterial and fungal samples were prepared and analyzed by scanning electron microscopy (SEM), transmission electron microscopy (TEM) and energy-dispersive X-ray spectroscopy (EDX). FIGS. 9A to 9I show SEM (FIGS. 9A, 9B, 9E and 9F), TEM (FIGS. 9D, 9G, 9H and 9I) and EDX (FIG. 9C) of E. coli coated with gold nanoparticles. FIG. 9G shows the E. coli. with AuNPs before the addition of TCEP and FIG. 911 shows it after the addition of TCEP. FIGS. 9J to 9Q show SEM (FIGS. 9J, 9K and 9N), TEM (FIGS. 9M, 9P and 9Q) and EDX (FIGS. 9L and 9O) of Lactobacillus coated with gold nanoparticles. FIGS. 9R to 9Y show SEM (FIGS. 9R, 9S, 9V and 9W), TEM (FIGS. 9U, 9X and 9Y) and EDX (FIG. 9T) of spores from Mucor coated with gold nanoparticles. The yellow patterns in the EDX mapping images shows the presence of gold and reveals the distribution of gold nanoparticles on the surface of microorganisms. The E. coli and Lactobacilus images were obtained after adjusting the pH of the reaction solution to 4. Nanocoating of the cells can be clearly observed in all microorganisms.

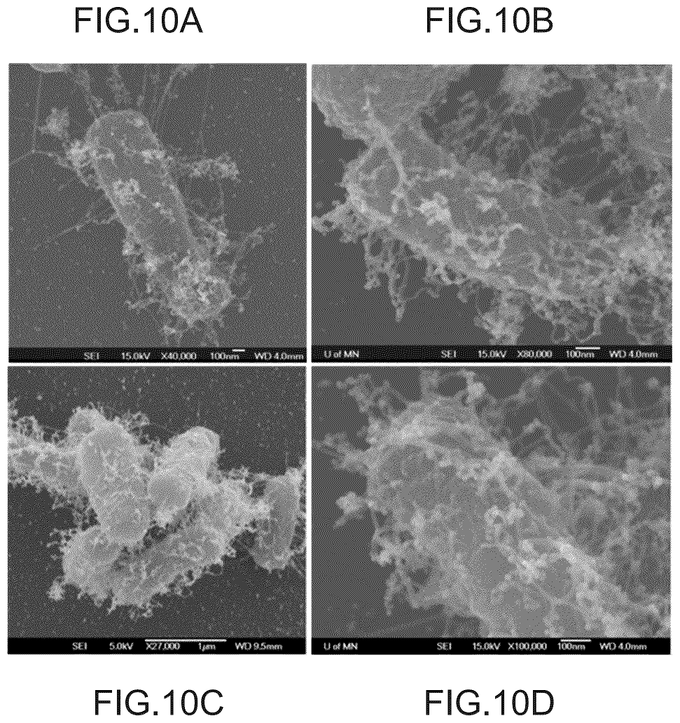

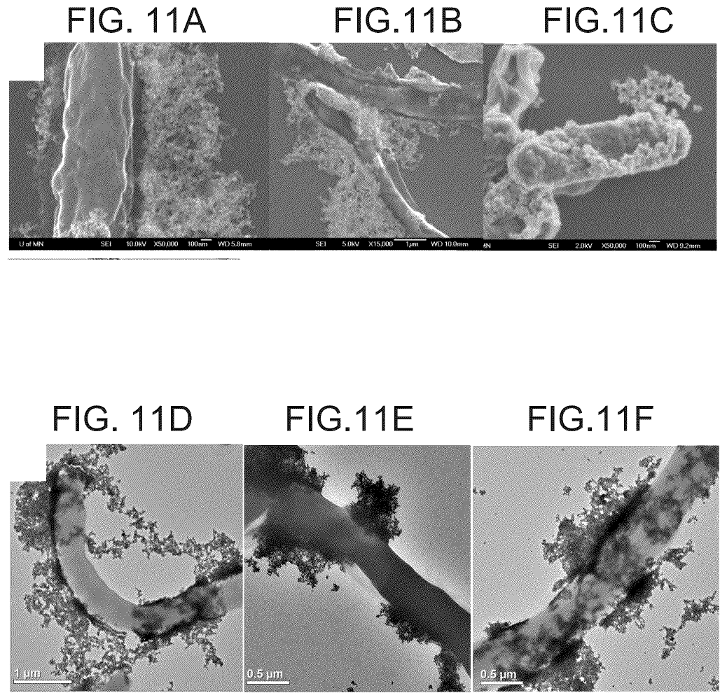

In addition, FIGS. 10A to 10D show that AuNPs assemble on bacterial pili as well. This is explained by the fact that these structures are composed of a Dsbc protein pilin, that usually contains two cysteine residues forming a disulfide bond. While fungal deacetylation results in highly stable nanocoatings due to the covalent bonding of chitin to other components in the fungal cell walls, bacterial reduction yields less stable samples. In fact, the Dsbc protein layers seem to easily peel-off after nanocoating, suggesting non-covalent bonding of Dsbc proteins layers to the bacterial cell wall (see FIGS. 11A to 11F). The images of nanocoated Lactobacillus were successfully obtained only after reducing the pH of the solution to 4, which likely strengthened electrostatic interactions between the Dsbc proteins and the bacterial cell wall. This challenging experiment reminds of the difficulty of observing and imaging microbial S-layers.

### Example 6: Plasmonic Detection Assays

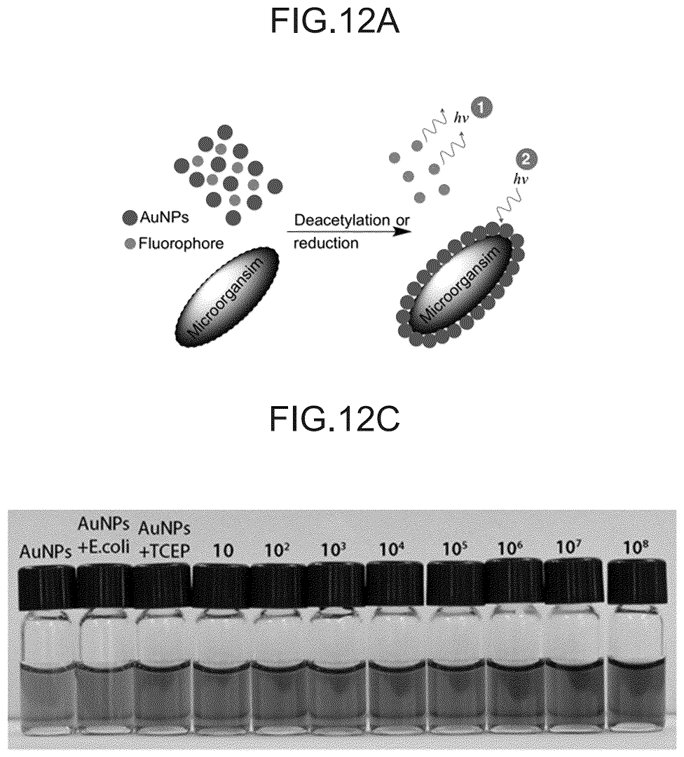

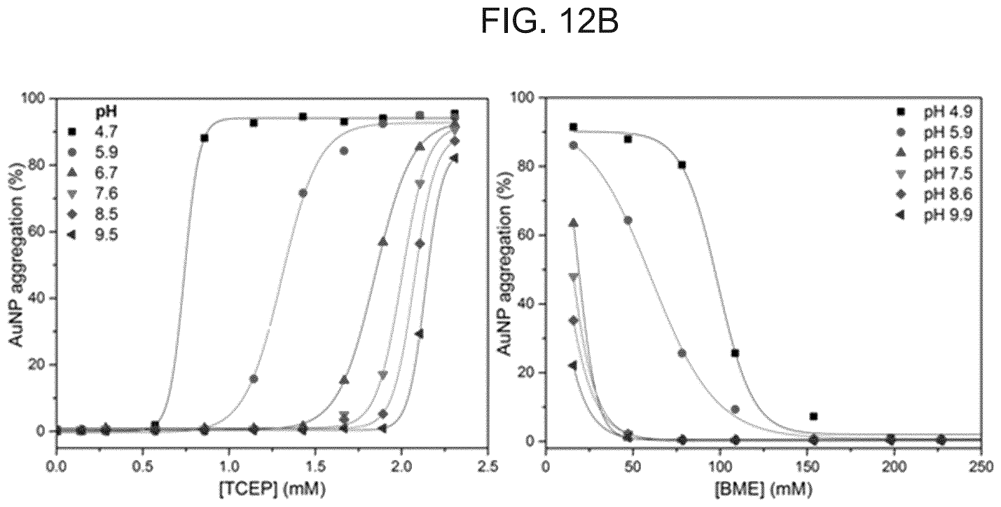

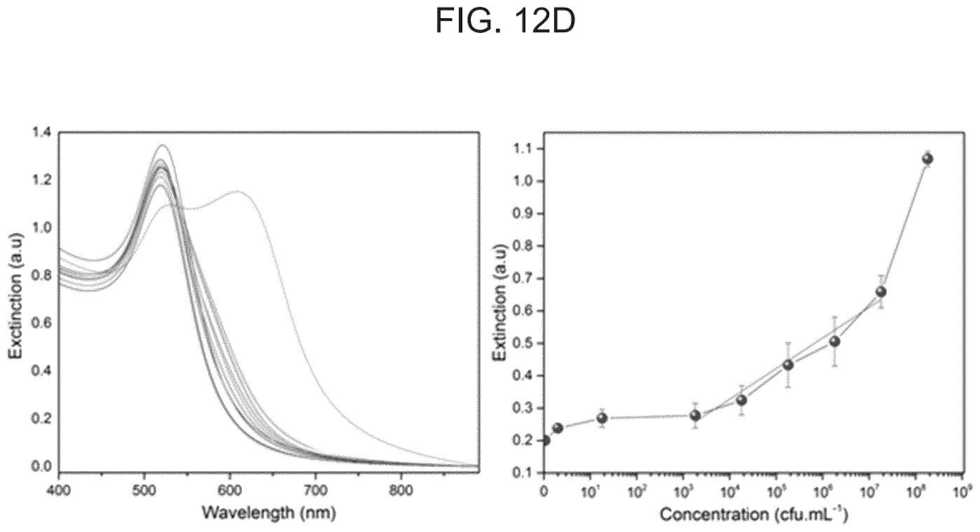

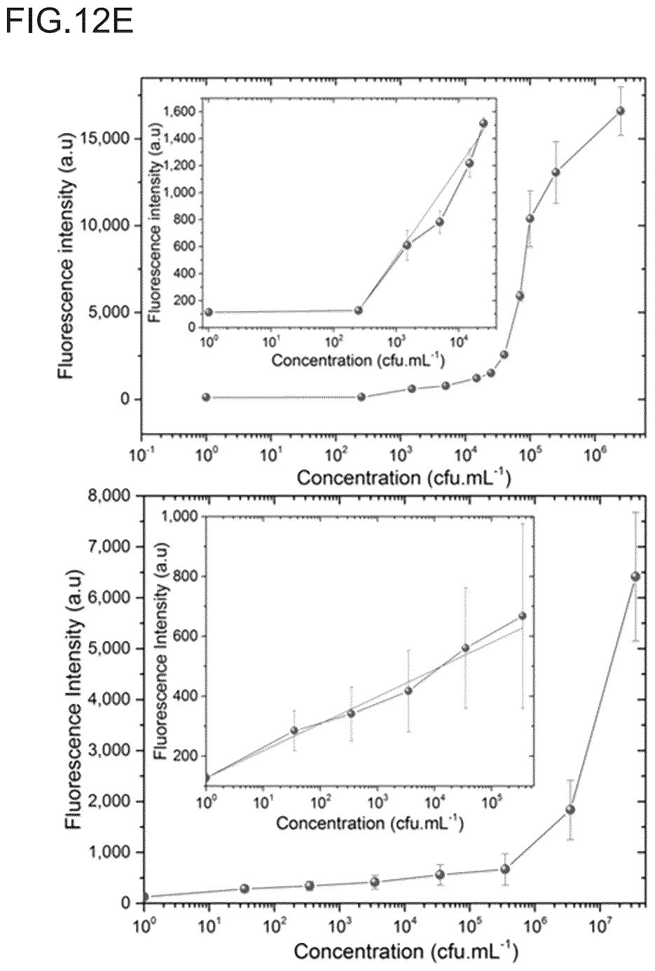

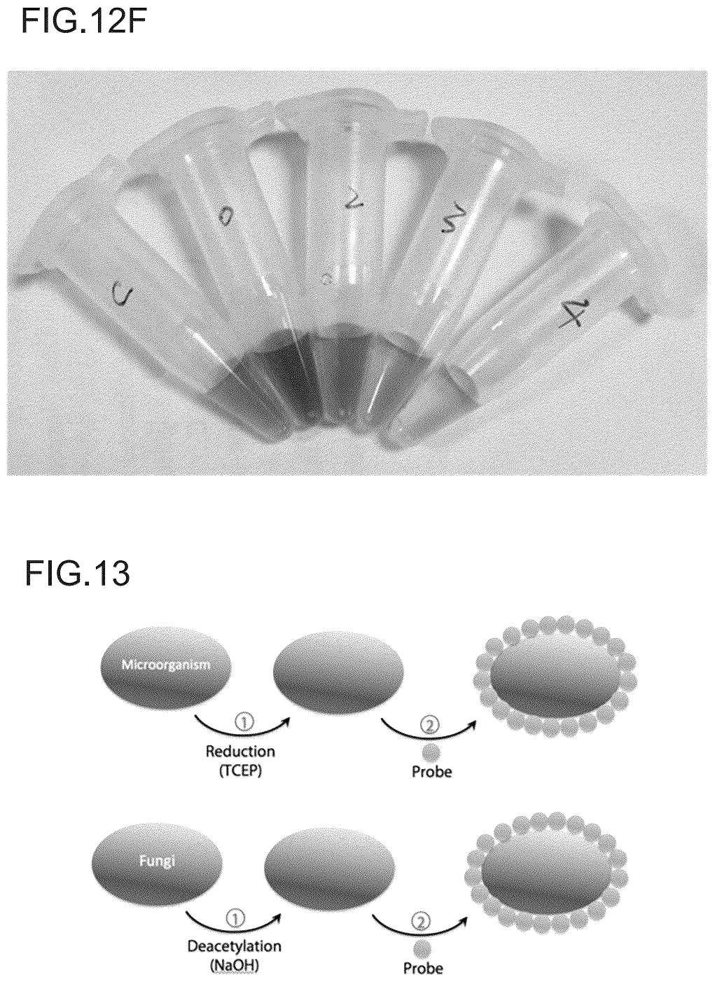

The assembly of AuNPs on the microbial surface can be used for microbial detection by monitoring a change in absorbance due to localized surface plasmon resonance. It can also be achieved by monitoring the change in fluorescence quenching of an aqueous fluorophore due to the presence of AuNPs (FIG. 12A). Since nanoparticle assembly is caused by reduction or deacetylation of the microbial surface layers using a reducing agent, it is important to first investigate the interaction of the reducing agent with both the nanoparticles and microorganisms. It is also important to know the effect of the pH conditions on these interactions. Different concentrations of two reducing agents; TCEP and 2-mercaptoethanol (BME) have been used at different pH to evaluate the impact of both parameters on the interaction. FIG. 12B show that concentrations below 1 mM for TCEP and over 100 mM for BME are suitable for detection assays at a pH around 6.5. The working concentration range for the reducing agent is a function of the pH conditions. Outside that range, assembly may be caused solely by the reducing agent by changing the zeta potential and disrupting the electrostatic repulsion between AuNPs. Because of its irreversible interaction with the disulfide bonds, TCEP is used here for the rest of the experiments. Since TCEP is not regenerated during the reaction, higher concentrations may be used for faster reduction of the microbial surface. Increasing the pH allows for an increase in TCEP concentration without causing nanoparticle assembly. However, the pH cannot be increased over 8 since the free thiol groups on the microbial surface will be deprotonated at higher pH, which hinders their interaction with the gold nanoparticles.

As depicted in FIG. 12C, microbial concentrations down to 10⁵ cfu.mL⁻¹ can induce a color change visible to the naked eye. The concentration of the nanoparticles may be reduced so that there will be no single nanoparticle left in solution after coating the cells, thus providing a better color shift for naked eye assessment (FIG. 12F). FIG. 12F shows the effect of the reduction of the concentration of the gold nanoparticles on the visual reading of the microbial load. The tube labeled “c” is a control (AuNP solution). The numbers on the tubes labeled 0, 2, 3 and 4 represent the dilution factor of the gold nanoparticle solution used for interaction with the microorganisms. Except the control, all tubes contain E. coli at a concentration of 10⁸ cfu.mL⁻¹. However, the zeta potential of the diluted AuNP solution should be kept the same as the original solution by diluting the nanoparticles in a citrate solution. To assess the limit of detection of the concept using localized surface plasmon resonance spectroscopy, different concentrations of microbial suspensions ranging from 10 to 10⁸ cfu.mL−1 were used for the assay. The absorption peak at around 600 nm (corresponding to assembled AuNPs) was analyzed with UV-visible spectroscopy (FIG. 12D), and cell concentration was verified using flow cytometry. The correlation between the peak intensity and the microbial concentration reveals a detection limit below 20 cfu.mL⁻¹ and quantification limit down to 10³ cfu.mL−1 for E. coli (FIG. 12D). Because the AuNPs assemble on the microbial surface, larger microorganisms are expected to have lower detection limits.

### Example 7: Fluorescence Detection Assay

To demonstrate the versatility of the detection concept, the same experiments were performed using fluorescence spectroscopy. In this case, AuNPs were mixed with a fluorophore, i.e Rhodamine 6G, and added to a microbial suspension. The presence of the AuNPs in solution quenches the fluorescence emission. The addition of TCEP and the subsequent assembly of the nanoparticles around the microorganisms leave the fluorophore alone in solution, leading to the enhancement of the fluorescence signal.

All fluorescence experiments were carried out with the GloMax® MultiJR fluorometer (Promega, Madison, WI) with an excitation wavelength of 525 nm. The gold nanoparticles and optical density (OD) of microbes were characterized using a UV-visible spectrophotometer (Shimadzu UV-1800, Shimadzu Corp., USA). Centrifugation was performed with microcentrifuge (MiniSpin Plus, Eppendorf™, USA). Extrusion of bacteria for fluorescence assays was achieved using the mini-extruder kit from Avanti Polar Lipds, Inc., (Alabster, AL). Raman and surface-enhanced Raman scattering analysis was performed using Witec alpha 300 R confocal Raman microscope with UHTS300 spectrometer and DV401 CCD detector (WITec Instruments, Corp., Knoxville, TN).

For fluorescence detection, the reduction of bacteria for 5 min is followed by removal of the TCEP from the sample using a mini-extruder. This step is required because TCEP interferes with the fluorescence signal of Rhodamine 6G. Then, 120 μL of the AuNP solution was added to 850 μL of reduced microbial samples. Next, 30 μL of freshly prepared 1 mM solution of Rhodamine 6G was added to the mixture and the fluorescence intensity was measured over time with a 3 s interval for a period of 3 minutes. Control sample measurements were performed using non-reduced or non-deacetylated microbial samples and AuNP-TCEP samples as controls.

The correlation between the microbial concentration and the fluorescence signal reveals a detection limit below 20 cfu.mL⁻¹ for E. coli and below 250 cfu.mL⁻¹ for Mucor fungi as seen in FIG. 12E. Both absorption and fluorescence transduction methods were performed on environmental samples. Similarly, to any other rapid microbial detection technique, the implementation of the concept to more complex samples requires upstream technologies to isolate the microorganisms from the matrix. Future work will focus on using surface phylogenetic markers to selectively target gram-positive or gram-negative bacteria.

One skilled in the art will appreciate that the methods described herein can be practiced with embodiments other than those disclosed. The disclosed embodiments are presented for purposes of illustration and not limitation. One will also understand that components of the methods depicted and described with regard to the figures and embodiments herein may be interchangeable.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise. The term “and/or” means one or all of the listed elements or a combination of any two or more of the listed elements.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising” and the like. For example, a conductive trace that “comprises” silver may be a conductive trace that “consists of” silver or that “consists essentially of” silver.

As used herein, “consisting essentially of,” as it relates to a composition, apparatus, system, method or the like, means that the components of the composition, apparatus, system, method or the like are limited to the enumerated components and any other components that do not materially affect the basic and novel characteristic(s) of the composition, apparatus, system, method or the like.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure, including the claims.

Also herein, the recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” a particular value, that value is included within the range.

Use of “first,” “second,” etc. in the description above and the claims that follow is not intended to necessarily indicate that the enumerated number of objects are present. For example, a “second” substrate is merely intended to differentiate from another infusion device (such as a “first” substrate). Use of “first,” “second,” etc. in the description above and the claims that follow is also not necessarily intended to indicate that one comes earlier in time than the other.

1-87. (canceled)

88. A method comprising:
adding a reducing activating reagent to a composition comprising a microorganism, the microorganism comprising an unmodified phylogenetic surface marker (PSM) on the surface of the microorganism, the PSM comprising a protein comprising a disulfide bridge;
allowing the activating reagent to modify the PSM to create an active PSM;
adding a probe to the composition, wherein the probe comprises a targeting portion, wherein the targeting portion does not include an antibody, and the targeting portion interacts with the active PSM to at least partially coat the microorganism with the probe.

89. The method according to claim 88, wherein the reducing agent comprises 2-mercaptoethanol (BME), 2-mercaptoethylamine-HCl (2-MEA-HCl), cysteine-HCl, dithiothreitol (DTT), tris(2-carboxyethyl)phosphine (TCEP), tris(2-carboxyethyl)phosphine hydrochloride (TCEP-HCl), or combinations thereof.

90. The method according to claim 88, wherein the probe further comprises an active agent.

91. The method according to claim 90, wherein the active agent comprises a plasmonic material, the plasmonic material comprising silver (Ag), copper (Cu), rhodium (Rh), Aluminum (Al), Zinc oxide (ZnO), platinum (Pt), palladium (Pd), Nickel (Ni), indium-tin-oxide (ITO), or combinations thereof.

92. The method according to claim 90, wherein the active agent comprises a magnetic material.

93. The method according to claim 92, wherein the magnetic material comprises iron, iron oxide, magnetite, or combinations thereof.

94. The method according to claim 90, wherein the active agent comprises a material having photothermal properties; a material having mechanical properties; a material having electrical properties; a material having optical properties; a material having antibiotic or biochemical properties; a material having fluorescent properties; a material having luminescent properties; a material having electrochemical properties; a material capable of acquiring fluorescent properties after interaction with the microorganism; a material capable of acquiring chromogenic properties after interaction with the microorganism; a material capable of catalyzing chromogenic reactions, fluorogenic reactions, electrogenic reactions, or a combination thereof; or combinations thereof.

95. The method of claim 1, further comprising separating the at least partially coated microorganism from the composition.

96. The method according to claim 95, wherein the step of separating the at least partially coated microorganisms from the composition comprises using a magnet, a magnetic field, or a combination thereof.

97. The method according to claim 95, further comprising detecting at least some portion of the at least partially coated microorganisms.

98. The method according to claim 97, wherein the step of detecting comprises detecting a change in color, a change in optical properties, a change in electrical signal, a change USE THEREOF in mechanical properties, a change in magnetic properties, a change in photothermal properties, or some combination thereof.

99. The method according to claim 95, further comprising capturing at least some portion of the at least partially coated microorganism.

100. The method according to claim 99 further comprising identifying, quantifying, concentrating, any combination thereof, the captured at least partially coated microorganisms.

101. A method comprising:
adding a deacetylation agent to a composition comprising a microorganism, the microorganism comprising unmodified chitin on the surface of the microorganism;
allowing the deacetylation agent to modify the chitin to create an active chitin;
adding a probe to the composition, wherein the probe comprises a targeting portion, wherein the targeting portion does not include an antibody, and the targeting portion interacts with the active chitin to at least partially coat the microorganism with the probe.

102. The method according to claim 101, wherein the deacetylating agent comprises lithium hydroxide (LiOH), sodium hydroxide (NaOH), potassium hydroxide (KOH), magnesium hydroxide (Mg(OH)₂), calcium hydroxide (Ca(OH)₂), or combinations thereof.

103. The method according to claim 101, wherein the probe further comprises an active agent.

104. The method according to claim 103, wherein the active agent comprises a plasmonic material, the plasmonic material comprising silver (Ag), copper (Cu), rhodium (Rh), Aluminum (Al), Zinc oxide (ZnO), platinum (Pt), palladium (Pd), Nickel (Ni), indium-tin-oxide (ITO), or combinations thereof.

105. The method according to claim 103, wherein the active agent comprises a magnetic material.

106. The method according to claim 105, wherein the magnetic material comprises iron, iron oxide, magnetite, or combinations thereof.

107. The method according to claim 103, wherein the active agent comprises a material having photothermal properties; a material having mechanical properties; a material having electrical properties; a material having optical properties; a material having antibiotic or biochemical properties; a material having fluorescent properties; a material having luminescent properties; a material having electrochemical properties; a material capable of acquiring fluorescent properties after interaction with the microorganism; a material capable of acquiring chromogenic properties after interaction with the microorganism; a material capable of catalyzing chromogenic reactions, fluorogenic reactions, electrogenic reactions, or a combination thereof or combinations thereof.

108. The method of claim 101, further comprising separating the at least partially coated microorganism from the composition.

109. The method according to claim 108, wherein the step of separating the at least partially coated microorganisms from the composition comprises using a magnet, a magnetic field, or a combination thereof.

110. The method according to claim 108, further comprising detecting at least some portion of the at least partially coated microorganisms.

111. The method according to claim 110, wherein the step of detecting comprises detecting a change in color, a change in optical properties, a change in electrical signal, a change in mechanical properties, a change in magnetic properties, a change in photothermal properties, or some combination thereof.

112. The method according to claim 108, further comprising capturing at least some portion of the at least partially coated microorganism.

113. The method according to claim 112, further comprising identifying, quantifying, concentrating, any combination thereof, the captured at least partially coated microorganisms.
